SEQUENCE LISTING (2 sequences)

SEQ ID NO: 1 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 2 - DNA, 882 bp, Staphylococcus aureus
gcagattctgatattaatattaaaaccggtactacagatattggaagcaatactacagtaaaaacaggtgatttagtcacttatgataaagaaaatggcatgcacaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaattaa

(19) United States
(12) Patent Application Publication
(10) Pub. No.: US 2023/0194499 A1
(43) Pub. Date: Jun. 22, 2023

(54) NANOPORE PREPARATION AND DETECTION METHOD AND DETECTION APPARATUS THEREOF

(71) Applicant: GENEUS TECHNOLOGIES CHENGDU ) CO., LTD., Chengdu, Sichuan (CN)

(72) Inventors: Yaozhong ZOU, Chengdu, Sichuan (CN); Yunpeng SU, Chengdu, Sichuan (CN); Peng JIANG, Chengdu, Sichuan (CN); Yan WANG, Chengdu, Sichuan (CN); Yuqin LI, Chengdu, Sichuan (CN)

(21) Appl. No.: 17/996,295

(22) PCT Filed: Apr. 14, 2021
(86) PCT No.: PCT/CN2021/087118

§ 371 Date: Oct. 14, 2022

(30) Foreign Application Priority Data
Apr. 14, 2020 (CN) 202010291055.8

(51) Int. Cl. G01N 33/487; C12Q 1/6869; G01N 33/68; B82Y 15/00; B82Y 5/00

(52) U.S. Cl. CPC G01N 33/48721; C12Q 1/6869; G01N 33/68; B82Y 15/00; B82Y 5/00

(57) ABSTRACT

Provided are a nanopore preparation and detection method and detection apparatus thereof. The method comprises: forming a nanopore by aggregating a plurality of protein monomers, with a signal detection region of the nanopore being formed in a narrow passage portion of the nanopore; and forming a positive charge cluster in the signal detection region of the nanopore, wherein the charge interaction between the positive charge cluster and negatively charged single molecule analytes that pass through the nanopore can lengthen the residence time of the single molecule analytes in the nanopore. Accordingly, the effective detection of analytes at a single-molecular level is realized, such that single molecule analytes that cannot generate an effective detection signal due to an interaction time with a. nanopore being too short can be effectively detected, the prevalence of single molecule analytes can be significantly improved, and different analytes and detection requirements are accommodated.

The Sequence Listing in ASCII text file format of 4,175 bytes in size, created on Oct. 14, 2022, with the file name “2022-10-14.Sequencelisting_ST25,” filed in the U.S. Patent and Trademark Office on even date herewith, is hereby incorporated herein by reference.

### FIELD OF THE INVENTION

The present disclosure belongs to the field of gene sequencing, and specifically, relates to a nanopore preparation and detection method detection device thereof.

### BACKGROUND OF THE INVENTION

Nanopores are a class of ion channels with a nanoscale. When a voltage is applied at the ends of a nanopore, electrolyte ions in solution pass through the nanopore to form a current under the driving force of an electric field. Some small molecules can interact with nanopores, usually in a way that they enter the nanopore channels and stay in them for a short time, thus affecting the magnitude of the current through the nanopore and generating a characteristic blocking current. Using the characteristic blocking current signal, nanopores can be used as a detector at the single-molecule level in different scenarios, including mechanistic studies of chemical reactions and sequence determination of nucleic acids.

However, nanopores still have significant limitations as single-molecule detectors. Analytes that can be detected by nanopores need to interact with the nanopore for a long time enough for their corresponding characteristic blocking current signals to be effectively captured and detected by the electronics, in addition to meeting certain size and charge property requirements. Various existing nanopores for nucleic acid sequencing, including protein nanopores such as α-bacterial toxin, MspA and CsgG or solid-state artificial nanopores prepared from materials such as graphene, are difficult to detect nucleotide signals directly at the single-molecule level. Nucleotide molecules, whether monophosphate, diphosphate or triphosphate, pass through the nanopore under the action of electric field driving force very fast, and the interaction time with the nanopore is usually only a few microseconds or even less. Even if the nucleotide causes a blocking current signal during this process, the too brief signal duration is well beyond the detection limits of all electronic components.

An existing improved solution is to obtain the ability to detect single molecule nucleotides by covalent modification of β-cyclodextrins at specific positions of α-bacterial toxins. However, the scheme using nanopores modified covalently with cyclodextrins has two aspects of obvious drawbacks: 1. Additional processes are required to prepare β-cyclodextrin that can be used to modify α-bacterial toxin, and because the modified sites are inside the pore of nanopore, the efficiency of the chemical reaction is difficult to guarantee, and the successful modified and unmodified nanopores are difficult to be effectively separated, which affects the subsequent detection; 2. The pore size of the modified nanopore is reduced from 1.4 nm to 0.8 nm, and the opening current is reduced to about 40% of that of the unmodified pore, which greatly narrows the signal window of the characteristic blocking current and narrows the detection range of the nanopore, and for Roche's sequencing system, since the nucleotides are labeled, too narrow nanopore aperture may cause the label to clog the nanopore, making the entire system inoperable.

Chinese patent CN102317310B proposes a method to enhance the translocation of charged analyte across transmembrane protein pore, which including increasing the frequency of nucleic acid chain translocation across said pore and decreasing the threshold voltage of said nucleic acid chain translocation across said pore by adding positive charges inside the nanopore, while this scheme can reduce the translocation speed of nucleic acid chain across said pore and increase the analyte residence time in the nanopore. However, in this patent, the sites where positive charges are introduced in the nanopore are scattered at various positions within the nanopore, with the main purpose of increasing the translocation frequency of the analyte and achieving nanopore capture efficiency of the analyte, and there are no data or examples demonstrating that the modified nanopore have the ability to detect single molecule nucleotides.

### SUMMARY OF THE INVENTION

The embodiments of the present disclosure provide a nanopore preparation and detection method and a detection device thereof for effective detection of single molecule analytes with improved detection accuracy.

In a first aspect, the embodiments of the present disclosure provide a nanopore preparation method comprising:

aggregating a plurality of protein monomers to form a nanopore, wherein the narrow portion of the channel of said nanopore forms a signal detection region of said nanopore;

forming a positive charge cluster in the signal detection region of said nanopore, the charge interaction between said positive charge cluster and a negatively charged single molecule analyte passing through said nanopore being capable of prolonging the residence time of said single molecule analyte within said nanopore.

In optional embodiments, said forming a positive charge cluster in the signal detection region of said nanopore comprises: introducing positively charged amino acid residues in the signal detection region of said nanopore by protein engineering to form said positive charge cluster.

In optional embodiments, said amino acid residues comprise: lysine, arginine or histidine.

In optional embodiments, said forming a positive charge cluster in the signal detection region of said nanopore comprises: introducing positively charged unnatural amino acids in the signal detection region of said nanopore by biochemical means to form said positive charge cluster.

In optional embodiments, said protein monomer comprises any one of α-hemolysin, MspA, CsgG, OmpF.

In optional embodiments, said single molecule analyte comprises nucleotides with different numbers of phosphates.

In a second aspect, the embodiments of the present disclosure provide a nanopore detection device comprising:

a test chamber having a nanopore and containing an electrolyte solution; and a detection circuit connected to said test chamber; wherein the narrow portion of the channel of said nanopore forms a signal detection region of said nanopore, said signal detection region having a positive charge cluster; when a negatively charged single molecule analyte in an electrolyte solution passes through said nanopore under the action of a driving voltage of the nanopore, a charge interaction between said positive charge cluster and the negatively charged single molecule analyte being capable of prolonging the residence time of said single molecule analyte within said nanopore.

In optional embodiments, the charge number of said positive charge cluster can be controlled to regulate the residence time of said single molecule analyte within said nanopore.

In optional embodiments, the driving voltage of said nanopore can be controlled to regulate the residence time of said single molecule analyte within said nanopore.

In optional embodiments, the concentration of said electrolyte solution can be controlled to regulate the residence time of said single molecule analyte within said nanopore.

In optional embodiments, said nanopore comprises a plurality of protein monomers.

In optional embodiments, said protein monomer comprises any one of α-hemolysin, MspA, CsgG, OmpF.

In optional embodiments, said single molecule analyte comprises nucleotides with different numbers of phosphates.

In a third aspect, the embodiments of the present disclosure provide a nanopore detection method used for the nanopore detection devices as described in the preceding embodiments, comprising:

controlling the number of charges of positive charge cluster in the signal detection region of the nanopore to regulate the residence time of the negatively charged single molecule analyte within said nanopore as it passes through said nanopore.

In optional embodiments, said controlling the number of charges of the positive charge cluster in the signal detection region of the nanopore to regulate the residence time of the negatively charged single molecule analyte within said nanopore as it passes through said nanopore comprises:

increasing the number of charges of the positive charge cluster in the signal detection region of said nanopore to prolong the residence time of the negatively charged single molecule analyte within said nanopore as it passes through said nanopore.

In optional embodiments, further comprising:

controlling the driving voltage of said nanopore to regulate the residence time of the negatively charged single molecule analyte within said nanopore as it passes through said nanopore.

In optional embodiments, said controlling the driving voltage of said nanopore to regulate the residence time of the negatively charged single molecule analyte within said nanopore as it passes through said nanopore comprises:

decreasing the driving voltage of said nanopore to prolong the residence time of the negatively charged single molecule analyte within said nanopore as it passes through said nanopore.

In optional embodiments, further comprising:

controlling the concentration of said electrolyte solution to regulate the residence time of the negatively charged single molecule analyte within said nanopore as it passes through said nanopore.

In optional embodiments, said controlling the concentration of said electrolyte solution to regulate the residence time of the negatively charged single molecule analyte within said nanopore as it passes through said nanopore comprises:

decreasing the concentration of said electrolyte solution to prolong the residence time of the negatively charged single molecule analyte within said nanopore as it passes through said nanopore.

In the embodiments of the present disclosure, positive charge cluster is introduced in the signal detection region of the nanopore to significantly prolong the interaction time between the negatively charged single molecule analyte and the nanopore through the interaction between the charges, allowing the characteristic blocking current signal detected in the signal detection region to be accurately detected by the electronic element. Compared with existing solutions, the embodiments of the present disclosure have at least the following beneficial effects: 1. Effective detection of analytes at the single-molecule level, especially for single-molecule nucleotides in scenarios related to nucleic acid sequencing, is achieved, and the detection rate of single-molecule analytes is significantly improved; 2. The nanopore prepared in the present disclosure for use in detection device can be flexibly adjusted according to the charge number of positive charge cluster, the driving voltage, and the concentration of the electrolyte solution, which makes the detection device applicable to different analytes and detection needs and has a wider application prospect.

### BRIEF DESCRIPTION OF THE DRAWINGS

In order to more clearly illustrate the technical solutions of the embodiments of the present disclosure or the prior art, a brief description of the drawings to be used in the description of the embodiments or the prior art will be given below. It will be apparent that the drawings described below are some embodiments of the present disclosure, and other drawings may be obtained from these drawings without inventive work to those of ordinary skill in the art.

FIGS. 1A and 1B are side and top views of a three-dimensional model of R4 nanopore prepared in accordance with embodiments of the present disclosure.

FIG. 2 is a schematic diagram of the structure of a nanopore detection device according to embodiments of the present disclosure.

FIG. 3A is a schematic diagram of the results of SDS electrophoresis detection of R4 nanopore.

FIG. 3B is a schematic diagram of the results of molecular sieve assay of R4 nanopore.

FIG. 4A is a plot of the signal detected by R4 nanopore for nucleotide triphosphateunder 100 mV and 400 mM KCl solution.

FIG. 4B is a plot of the signal detected by natural a-hemolysin for nucleotide triphosphate under the same conditions.

FIG. 4C is a statistical analysis of the signal detected by the R4 nanopore for nucleotide triphosphate.

FIG. 4D is a plot of the signal detected by R4 nanopore for nucleotide monophosphate.

FIG. 5A is a plot of the signal detected by R5 nanopore for nucleotide triphosphate under 100 mV and 300 mM KCl solution.

FIG. 5B is a plot of the signal detected by R4 nanopore for nucleotide triphosphate under the same conditions.

FIGS. 6A-6C are plots of the signals detected by R4 nanopore for nucleotide triphosphate at the concentrations of 1 M KCl, 500 mM KCl, and 300 mM KCl solutions, respectively.

FIGS. 7A-7F are plots of the signals detection by R4 nanopore for nucleotide triphosphate at voltages of 180 mV, 160 mV, 140 mV, 120 mV, 100 mV, and 80 mV, respectively.

### EMBODIMENTS

In order to make the purpose, technical solutions and advantages of the embodiments of the present disclosure clearer, the technical solutions of the embodiments of the present disclosure will be clearly and completely described below in conjunction with the drawings of the embodiments of the present disclosure. Obviously, the described embodiments are part of the embodiments of the present disclosure, and not all of them. Based on the embodiments in the present disclosure, all other embodiments obtained by those persons of ordinary skill in the art without inventive word fall within the scope of protection of the present disclosure.

In this disclosure, it is understood that terms such as “comprise” or “have” are intended to indicate the presence of the features, figures, steps, actions, parts, portions, or combinations thereof disclosed in this specification, and are not intended to exclude the possibilities that one or more other features, figures, steps, actions, parts, portions, or combinations thereof may exist or be added.

As previously mentioned, existing nanopores for analyte detection are deficient in their ability to detect single molecule analytes. In particular, effective detection of nucleotides at the single molecule level is important for developing new nucleic acid sequencing protocols or improving the accuracy of existing sequencing protocols.

The embodiments of the present disclosure first propose a nanopore preparation method that aggregates a plurality of protein monomers to form a nanopore, wherein the narrow portion of the channel of said nanopore forms a signal detection region of said nanopore; a positive charge cluster is formed in the signal detection region of said nanopore, and a charge interaction between said positive charge cluster and a negatively charged single molecule analyte passing through said nanopore is capable of prolonging the residence time of said single molecular analyte within said nanopore. In the method, a positive charge cluster is formed in the signal detection region (constriction site) of the nanopore, and when a negatively charged single molecule analyte enters the nanopore under the action of an electric field, the charge interaction between the positive charge cluster and the negatively charged single molecule analyte significantly prolongs the residence time of the single molecule analyte in said nanopore, thereby increasing the duration of the characteristic current signal of blockade in the nanopore. When the nanopore with such characteristics prepared by this method is used in the detection device, the residence time of the single-molecule analyte passing through the nanopore is significantly prolonged, and the residence time can reach the millisecond level, so that the characteristic blocking current signal generated in the nanopore can be effectively captured by the electronic element, so that the single-molecule analyte that would otherwise be undetectable due to the short residence time can be effectively detected, and the detection rate of the single-molecule analyte can be improved.

In some embodiments, said nanopore comprise protein nanopore. Protein nanopore that can be used include, but are not limited to, bacterial toxin α-hemolysin, MspA, CsgG, OmpF, etc. These protein nanopores are widely used in numerous scenarios for molecular detection, including sequencing, and have known high-resolution three-dimensional structures that allow easy localization of their signal detection regions and nearby amino acid residues. Therefore, the introduction of positively charged mutations in the signal detection regions of these nanopores by genetic engineering means is highly targeted and feasible.

In some embodiments, the single molecule analyte may include, but are not limited to, nucleotides with different numbers of phosphates.

The nanopore of the bacterial toxin α-hemolysin is taken as an example. The bacterial toxin α-hemolysin is a natural protein monomer containing 293 amino acids and has the following protein primary structure sequence.

(SEQ ID NO: 1)
ADSDINIKTGTTDIGSNTTVKTGDLVTYDKENGMHKKVFYSFIDDKNHN
KKLLVIRTKGTIAGQYRVYSEEGANKSGLAWPSAFKVQLQLPDNEVAQI
SDYYPRNSIDTKEYMSTLTYGFNGNVTGDDTGKIGGLIGANVSIGHTLK
YVQPDFKTILESPTDKKVGWKVIFNNMVNQNWGPYDRDSWNPVYGNQLF
MKTRNGSMKAADNFLDPNKASSLLSSGFSPDFATVITMDRKASKQQTNI
DVIYERVRDDYQLHWTSTNWKGTNTKDKWTDRSSERYKIDWEKEEMTN

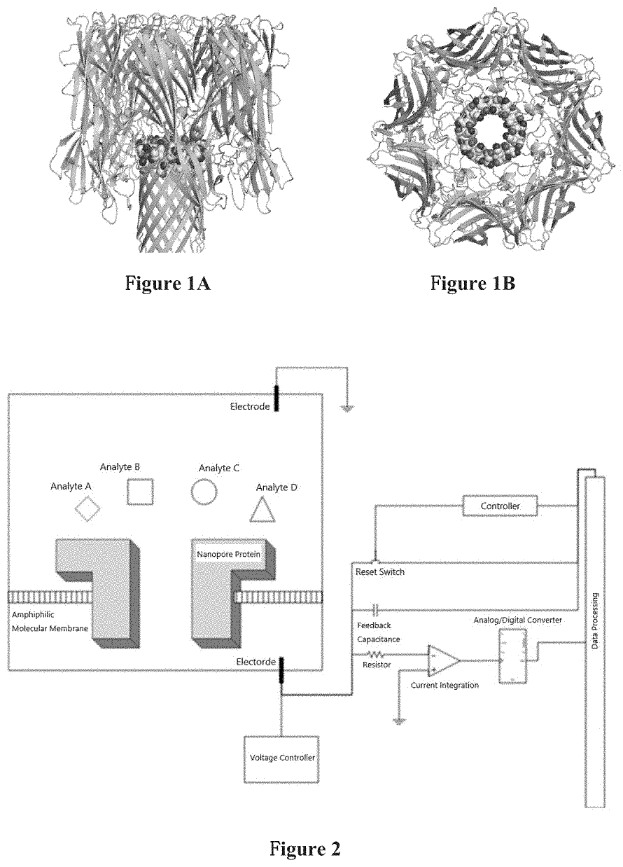

Seven protein monomers of α-hemolysins spontaneously aggregate to form a nanopore in the presence of detergents, phospholipids, or other amphiphilic molecules, and the side and top views of its three-dimensional model structure is shown in FIGS. 1A and 1B, respectively. Wherein, the narrowest part of the nanopore channel consisting of E111, M113, and K147 of seven monomers, represented by the spherical model in FIGS. 1A and 1B, is the signal detection region (constriction site) where the analyte causes the characteristic blocking current.

In some embodiments, positively charged mutations of amino acid residues, such as, but not limited to, E111R, M113R, T115R, G143R, or any combination of these mutations, is introduced in and near this signal detection region by means of protein engineering, and the nanopore formed by the mutants will have a positive charge cluster with a high positive charge density formed in the vicinity of the signal detection region. The positively charged mutation on each protein monomer corresponds to seven additional positive charges on the nanopore. In some embodiments, the amino acid residues can include: lysine (Lys), arginine (Arg), or histidine (His) protonated at low pH. In some embodiments, mutant with multiple mutation sites, such as E111R, M113R, T115R, G143R, K147R, can be prepared with the same means as the wild-type protein to obtain highly pure heptameric nanopores with positive charge cluster.

In other embodiments, protein monomers with corresponding positively charged mutations can be mixed with protein monomers without mutations to form heteropolymer, thus allowing finer regulation of the positive charge density in the signal detection region of the nanopore, rather than only the case where 7 positive charges are added for each mutation introduced. For example, three wild-type α-hemolysin monomers and four monomers with the M113R mutation form a heteroheptamer that will carry four positive charges at position 113 within the nanopore.

In other embodiments, non-natural amino acids can also be introduced in the signal detection region of the nanopore by specific biochemical means. The non-natural amino acids can be different from the natural lysine (Lys), arginine (Arg), or histidine (His), but can carry a different number of charges, thus allowing the nanopore formed by the protein monomers into which they are introduced to have an adjustable positive charge cluster with more abundant charges.

The embodiments of the present disclosure also propose a nanopore detection device. After the preparation of the nanopore with positive charge cluster, the modified nanopore protein can be embedded in a specific phospholipid bilayer or an artificial bilayer with similar physicochemical properties to form a nanopore detection device as shown in FIG. 2. The modified nanopore protein is purified by biochemical means similar to wild nanopore and can retain its biochemical activity well. The purified and prepared nanopore protein can be spontaneously embedded in the phospholipid bilayer or the artificial bilayer due to its lipophilic properties.

The nanopore detection device comprises a test chamber embedded with the prepared nanopore and a microcurrent detection circuit connected thereto. The test chamber comprises a first compartment and a second compartment separated by a phospholipid bilayer or an artificial bilayer membrane, the first compartment and the second compartment contain an electrolyte solution, the analyte is located in the electrolyte solution in the first compartment, and the negatively charged analyte, such as but not limited to nucleotide monophosphate (NMP or dNMP), nucleotide bisphosphonate (NDP or dNDP), nucleotide triphosphates (NTP or dNTP), polyphosphate nucleotide (with four or more phosphate groups), and some nucleotide derivatives pass from one side of the bilayer membrane through the nanopore into the second compartment on the other side. During this process, the negatively charged analyte is attracted and captured by the positive charge cluster introduced in the signal detection region of the nanopore, thus staying longer and allowing the generated characteristic blocking current signal to be effectively captured by the microcurrent detection circuit.

Electrodes at both ends of the test chamber can be utilized to apply a voltage in the nanopore detection device of the embodiments of the present disclosure, for example, 50-300 mV, including but not limited to 75-275 mV, 100-250 mV, 125-225 mV, 150-200 mV, or 100 mV, 125 mV, 150 mV, 175 mV, 200 mV, thereby creating potential gradient across the membrane along the inner diameter of the nanopore. The electrolyte is driven by the potential gradient and moves in a directional manner within the nanopore to form a current. When the negatively charged analyte, including nucleotide molecules with different numbers of phosphates, approachs the nanopore opening under the dual action of free diffusion and electrophoretic motion, it will be captured by the potential gradient along the nanopore and thus driven by the electric field force to pass through the nanopore. For most of the natural nanopore proteins, including a-hemolysin, MspA, CsgG, OmpF, etc., the time of nucleotide molecule passing through is in the range of 1-10 μs, and the caused blocking current signal is only in the order of pA. Such weak signal strength and short signal duration cannot support effective detection by the electronic components. In the nanopore detection device of the embodiments of the present disclosure, a positive charge cluster is introduced in the nanopore protein, and the positive charge cluster with high density can effectively slow down the translocation of the negatively charged analyte through the nanopore, prolonging the signal duration and allowing the characteristic blocking current signal of otherwise undetectable single-molecule analytes to be effectively captured, thereby obtaining the detection capability of single-molecule analytes. In particular, the nanopore with single-molecule nucleotide detection capability can be used in different detection scenarios, including for sequencing or to assist in improving sequencing accuracy.

In some embodiments, the phospholipid constituting the phospholipid bilayer may comprise 1,2-diphytanoyl-sn-glycero-3-phosphocholine (DPhPC). However, the embodiments of the present disclosure are not limited to specific phospholipids, including but not limited to some other choline-based phospholipids or artificial amphiphilic molecules, which can be used in the embodiments of the present disclosure as long as they can divide the electrolyte solution pool into two parts with separate electrodes and electrically isolated from each other, and can support the structure of nanopore protein to form a stable current channel.

The embodiments of the present disclosure also propose a nanopore detection method suitable for the nanopore detection device described above. The method includes controlling the number of charges of the positive charge cluster in the signal detection region of the nanopore to regulate the residence time of the negatively charged single molecule analyte within said nanopore as it passes through said nanopore.

In some embodiments, increasing the number of charges of the positive charge cluster in the signal detection region of the nanopore can significantly prolong the corresponding signal duration of the analyte passing through the nanopore. For example, adding one more positive charge to the preferred position of the monomer of a-hemolysin results in a corresponding increase of seven positive charges on the heptamer nanopore. The positive charge cluster with more charges have a significantly enhanced ability to capture the negatively charged analyte. In some embodiments, the time for nucleotide triphosphate to pass through the nanopore with more positive charges can be further prolonged by close to one order of magnitude.

In some embodiments, the method can further include controlling the driving voltage of said nanopore to regulate the residence time of the negatively charged single molecule analyte within said nanopore as it passes through said nanopore.

In some embodiments, decreasing the driving voltage of said nanopore can prolong the residence time of the negatively charged single molecule analyte within said nanopore as it passes through said nanopore. For example, by reducing the driving voltage from 180 mV to 120 mV, 100 mV, or 80 mV, the negatively charged analyte, such as nucleotide, is subjected to significantly less electric field driving force and the time it translocates through the nanopore increases significantly thereafter. In some embodiments, the driving voltage is reduced from 180 mV to 80 mV, and the corresponding signal duration for nucleotide translocation through the nanopore can be significantly prolonged from less than 100 μs to more than 1 ms, allowing for more efficient detection of the analyte.

In some embodiments, the method can further include controlling the concentration of electrolyte solution of said detection device to regulate the residence time of the negatively charged single molecule analyte in said nanopore as it passes through said nanopore.

In some embodiments, decreasing the concentration of the electrolyte solution of said detection device can prolong the residence time of the negatively charged single molecule analyte within said nanopore as it passes through said nanopore. For example, potassium chloride solution is a electrolyte solution commonly used in nanopore assays. The concentration of the potassium chloride solution has a very significant effect on the duration of analyte translocation through the nanopore. In some embodiments, simply decreasing the potassium chloride concentration from 1 M to 300 mM is sufficient to prolong the time of translocation of nucleotide triphosphate through the nanopore by more than one order of magnitude.

The nanopore detection method of the embodiments of the present disclosure demonstrates the ability to effectively regulate the target signal duration of the analyte, thereby enriching the detection means and corresponding signal characteristics, allowing the nanopore detection of analytes represented by single-molecule nucleotides to be applied to more diverse scenarios and adapted to different analytes and detection needs.

The present disclosure is further described below in connection with examples of specific applications.

### EXAMPLE 1: PREPARATION OF α-HEMOLYSIN MUTANT

In this example, the natural α-hemolysin gene was obtained by direct synthesis based on the Genebank Accession No. NC_007795.1, and the cDNA sequence synthesized was as follows.

(SEQ ID NO: 2)
gcagattctgat attaatatta aaaccggtac tacagatatt
ggaagcaata ctacagtaaa aacaggtgat ttagtcactt atgataaaga aaatggcatg
cacaaaaaag tattttatag ttttatcgat gataaaaatc ataataaaaa actgctagtt
attagaacga aaggtaccat tgctggtcaa tatagagttt atagcgaaga aggtgctaac
aaaagtggtt tagcctggcc ttcagccttt aaggtacagt tgcaactacc tgataatgaa
gtagctcaaa tatctgatta ctatccaaga aattcgattg atacaaaaga gtatatgagt
actttaactt atggattcaa cggtaatgtt actggtgatg atacaggaaa aattggcggc
cttattggtg caaatgtttc gattggtcat acactgaaat atgttcaacc tgatttcaaa
acaattttag agagcccaac tgataaaaaa gtaggctgga aagtgatatt taacaatatg
gtgaatcaaa attggggacc atatgataga gattcttgga acccggtata tggcaatcaa
cttttcatga aaactagaaa tggctctatg aaagcagcag ataacttcct tgatcctaac
aaagcaagtt ctctattatc ttcagggttt tcaccagact tcgctacagt tattactatg
gatagaaaag catccaaaca acaaacaaat atagatgtaa tatacgaacg agttcgtgat
gactaccaat tgcactggac ttcaacaaat tggaaaggta ccaatactaa agataaatgg
atagatcgtt cttcagaaag atataaaatc gattgggaaa aagaagaaat gacaaattaa

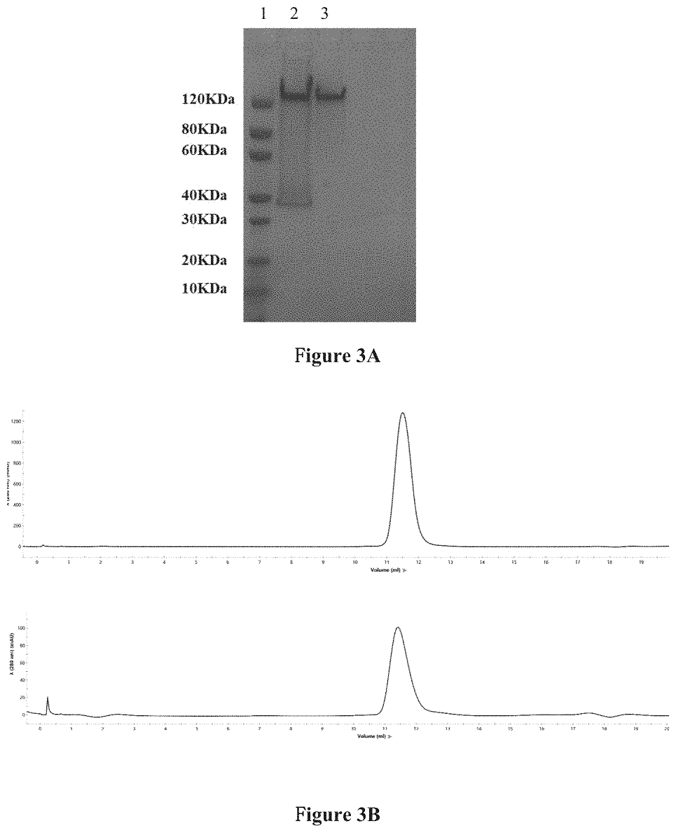

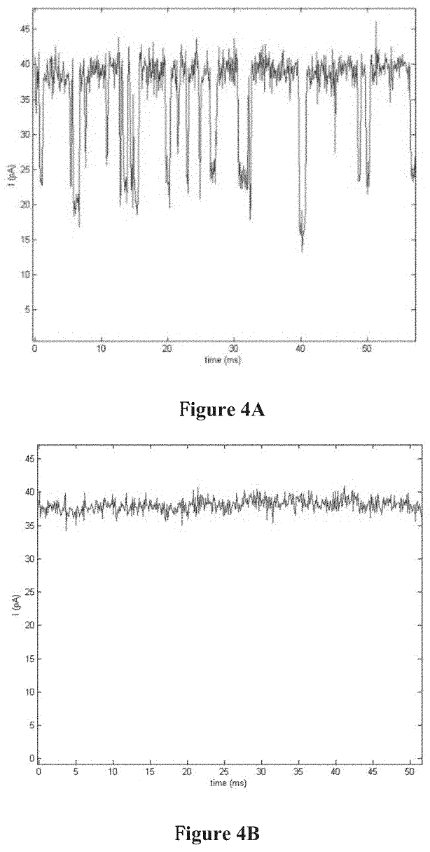

The synthesized α-hemolysin is cloned directly into the expression vector pET26b. Targeted mutations are introduced in the above gene using Agilent's quick change kit to prepare a mutant with four mutations, E111R/M113R/T115R/K143R, named R4 nanopore. The structure of R4 nanopore is shown in FIG. 1, and all the mutations are concentrated near the signal detection region (constriction site) of α-hemolysin. R4 nanopore can be obtained in the same way as the natural a-hemolysin nanopore, and the results of SDS electrophoresis of its heptamer are shown in FIG. 3A, and the results of molecular sieve assay are shown in FIG. 3B. Lane 1 in FIG. 3A represents the standard molecular weight marker, lane 2 indicates that a few heptamers of R4 mutants are dissociated into monomers during electrophoresis, and lane 3 represents the wild-type a-bacterial toxin. In FIG. 3B, the molecular sieve assay of purified R4 nanopore is shown on the upper side, and the molecular sieve profile of wild-type a-bacterial toxin is shown on the lower side as a control. In a similar way, mutants with more positive charges, E111R/M113R/T115R/G143R/K147R, can also be prepared, which can be named as R5 nanopore.

On the basis of the preparation of the modified nanopore, a compartment system with test chambers that meets the requirements can be selected, for example, a compartment system with an opening pore size of 50 μm between the left and right compartments and a hydrophobic material suitable for phospholipid attachment near the opening. 0.3 M KCl solution is added to each of the left and right compartments with the liquid level below the opening. 1,2-diphytanoyl-sn-glycero-3-phosphocholine (DphPC), dissolved in organic solvent pentane at a final concentration of 1 mg/ml, is selected. This phospholipid solution is added to the KCl solution already present in the compartment to form an organic phase covering the KCl liquid surface. By adding 0.3 M KCl solution along the outer wall of the compartment, the organic phase containing the phospholipid layer will rise with the liquid level over the small pore between the two compartments. During this process, the phospholipid binds to the hydrophobic material near the pore and spontaneously forms a phospholipid bimolecular membrane across the pore.

R4 nanopore solution is added to the first compartment to a final concentration of 1 nM, while the current between the two compartments is tested. When the single molecule nanopore is spontaneously inserted into the phospholipid bilayer, the system will detect the open pore current of R4. The excess R4 nanopore solution is removed in time to ensure that the phospholipid membrane has only single-molecule R4 nanopore channel. As a result, phospholipid membrane with R4 nanopore protein can be prepared and assembled into the nanopore detection device of the embodiments of the present disclosure.

After obtaining a single channel of R4 nanopore, the driving voltage is set to 180 mV, and the opening current through R4 nanopore is recorded to be ˜106 pA. Compared with the opening current of ˜110 pA under the same conditions of wild-type α-hemolysin, there is no significant difference, indicating that R4 protein, which is deeply modified and introduced 28 positive charges near the signal detection region, still retains a similar current detection window as the natural α-hemolysin. In contrast, the opening current of the a-hemolysin covalently modified with β-cyclodextrin is reduced to about 40% of that of the unmodified nanopore, which is only ˜45 pA.

### EXAMPLE 2: CAPTURE OF NUCLEOTIDES WITH DIFFERENT PHOSPHATE BY R4 NANOPORE

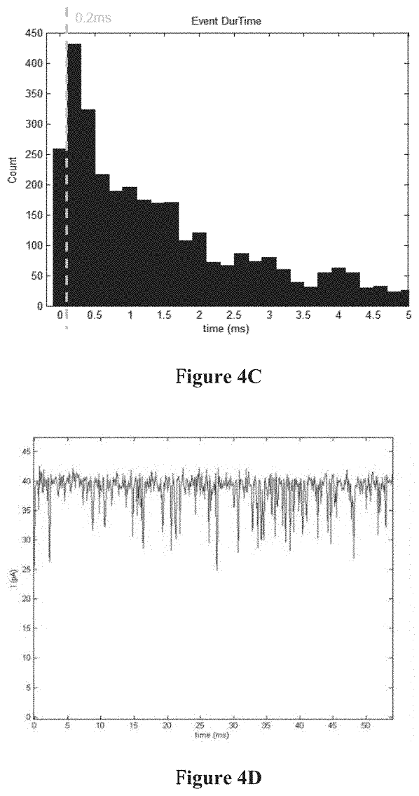

A final concentration of 1 μM nucleotide triphosphate (dCTP) is added to the electrolyte solution pool in the first compartment of the nanopore detection device assembled with R4 nanopore. The voltage is set to 100 mV, and under the electric field force, dCTP is captured by R4 nanopore and translocated from the first compartment to the second compartment. FIG. 4A shows the signal detected by R4 mutant for nucleotide triphosphate under 100 mV voltage and 400 mM KCl solution. It can be seen that dCTP interacts with R4 nanopore to give a clear signal of blocking current. As a control, as shown in FIG. 4B, in the nanopore detection device composed of natural α-hemolysin with the addition of dCTP in the first compartment, the system is unable to detect an effective dCTP blocking current signal.

Statistical analysis of the duration of the dCTP current signal captured by R4 shows that the signal duration conforms to an exponential distribution, which is in perfect agreement with the theoretically predicted model. FIG. 4C shows the statistical analysis of the nucleotide triphosphate signal detected by R4, with a random exponential distribution of the signal density over different durations.

Similar to dCTP, the addition of 1 μM of nucleotide monophosphate (dCMP) to the first compartment of R4 nanopore also results in the detection of a significant characteristic blocking current signal for the analyte. FIG. 4D shows the signal detected by R4 mutant for nucleotide monophosphate, where the signal duration is significantly shorter than the corresponding signal duration for nucleotide triphosphate due to the lesser charge of the analyte.

### EXAMPLE 3: CAPTURING OF DCTP BY R5 NANOPORE WITH MORE POSITIVELY CHARGED MUTATIONS

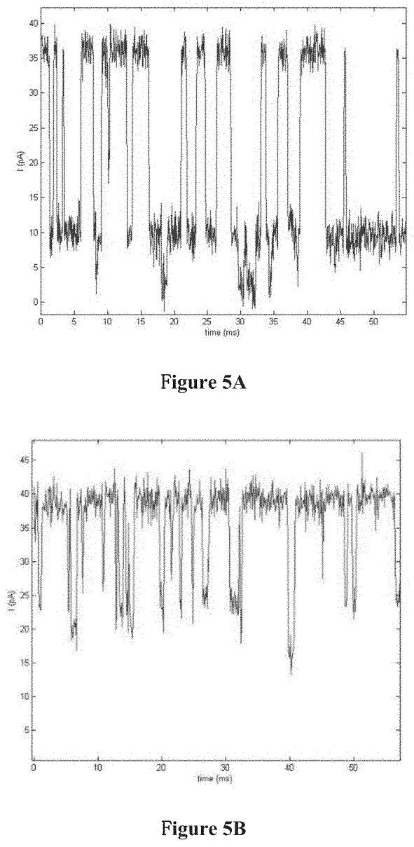

As described in the above examples, R4 nanopore is replaced with R5 nanopore in the same way, and the blocking current generated by R5 nanopore capturing nucleotide triphosphate dCTP is recorded under the same conditions. FIG. 5A shows the signal of nucleotide triphosphate detected by R5 nanopore under 100 mV voltage and 300 mM KCl solution, and FIG. 5B shows the signal of nucleotide triphosphate detected by R4 nanopore under the same conditions. As the R5 nanopore has more introduced positive charges near the signal detection region, its interaction force with dCTP is significantly increased and the duration of the blocking current signal caused by dCTP is significantly increased. It can be seen that R5 mutant with more positive charges has a significantly longer signal duration and deeper blocking current amplitude for the capture detection of nucleotide triphosphate than that detected with R4 nanopore.

### EXAMPLE 4: EFFECT OF SALT CONCENTRATION ADJUSTMENT ON THE DURATION OF DCTP SIGNAL

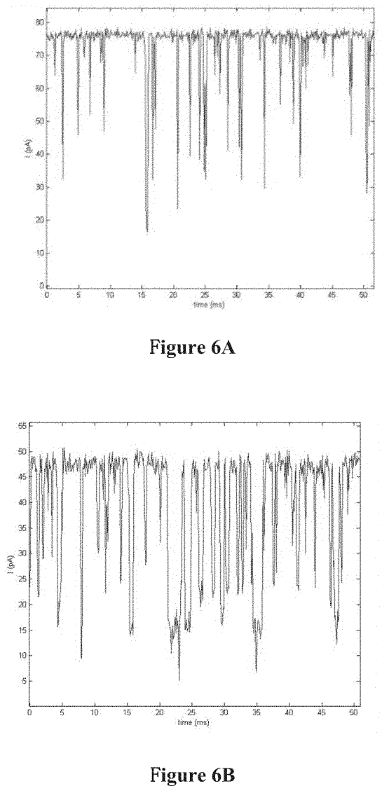

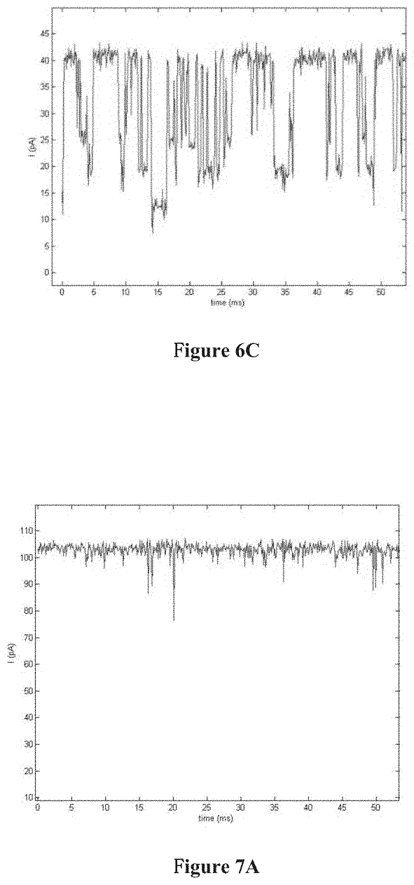

The salt concentration of KCL in the solution pool in the nanopore detection device is adjusted between 0.3M-1M and the blocking current caused by dCTP is recorded, respectively. The corresponding signal data obtained are shown in FIGS. 6A-6C. FIGS. 6A-6C show the results of the signal detected by R4 nanopore for dCTP at 100 mV and at 1 M KCl, 500 mM KCl, and 300 mM KCl solution concentrations, respectively. The results show that the signal duration of dCTP was inversely correlated with the salt concentration, and the lower the salt concentration, the longer the signal duration.

### EXAMPLE 5: EFFECT OF DRIVING VOLTAGE ADJUSTMENT ON THE DURATION OF DCTP SIGNAL

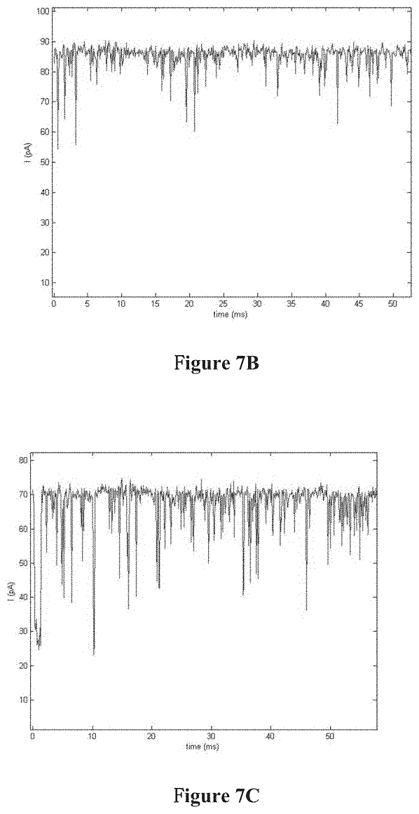

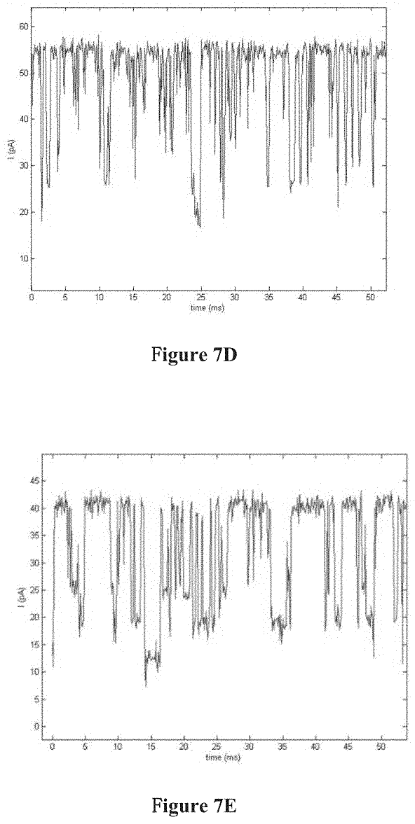

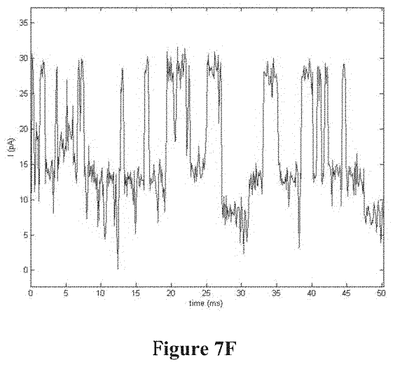

Different driving voltages are applied to the nanopore detection device, such as 60 mV, 70 mV, 80 mV, 90 mV, 100 mV, . . . , 180 mV, 190 mV, 200 mV, etc., and the blocking currents generated by the capturing of dCTP by R5 nanopore are recorded respectively. FIGS. 7A-7F show the effect of different voltages on the signal detected by R4 nanopore for nucleotide triphosphate at 300 mM salt concentration of 180 mV, 160 mV, 140 mV, 120 mV, 100 mV and 80 mV, respectively. The results show that the signal duration was very sensitive to the driving voltage, the lower the voltage, the longer the signal duration. At <100 mV, the signal duration could be several milliseconds or even tens of milliseconds. At greater than 150 mV, the signal duration is already very short, with only a few sporadic signal spikes.

The above examples and related data detail the implementation and practical effects of the present disclosure. The highly adjustable duration of the characteristic current signal of the analyte also makes the specific application scenario of the present disclosure broader. It should be noted that the present disclosure is not limited to the particular examples described herein, which are intended as exemplary illustrations of various aspects of the present disclosure. Many modifications and variations can be made to the present disclosure without departing from the spirit and scope of the present disclosure, as will be apparent to those skilled in the art. Functionally equivalent methods and apparatus within the scope of the present disclosure, other than those enumerated herein, will become apparent to those skilled in the art from the foregoing description. These modifications and variations are intended to fall within the scope of the appended claims. The present disclosure is limited only by the terms of the appended claims and the full scope of the equivalents of those claims. It should be understood that the present disclosure is not limited to particular methods, reagents, compound compositions, or biological systems that are of course subject to variation. It should also be understood that the terminology used herein is used only for the purpose of describing particular examples and is not limiting.

1. A nanopore preparation method, characterized in that, the method comprises:
aggregating a plurality of protein monomers to form a nanopore, wherein the narrow portion of the channel of said nanopore forms a signal detection region of said nanopore;
forming a positive charge cluster in the signal detection region of said nanopore, the charge interaction between said positive charge cluster and a negatively charged single molecule analyte passing through said nanopore being capable of prolonging the residence time of said single molecule analyte within said nanopore.

2. The method according to claim 1, characterized in that, said forming a positive charge cluster in the signal detection region of said nanopore comprises: introducing positively charged amino acid residues in the signal detection region of said nanopore by protein engineering to form said positive charge cluster.

3. The method according to claim 2, characterized in that, said amino acid residues comprise: lysine, arginine or histidine.

4. The method according to claim 1, characterized in that, said forming a positive charge cluster in the signal detection region of said nanopore comprises: introducing positively charged unnatural amino acids in the signal detection region of said nanopore by biochemical means to form said positive charge cluster.

5. The method according to claim 1, characterized in that, said protein monomer comprises any one of a-hemolysin, MspA, CsgG, OmpF.

6. The method according to claim 1, characterized in that, said single molecule analyte comprises nucleotides with different numbers of phosphates.

7. A nanopore detection device, characterized in that, the device comprises:
a test chamber having a nanopore and containing an electrolyte solution; and
a detection circuit connected to said test chamber; wherein the narrow portion of the channel of said nanopore forms a signal detection region of said nanopore, said signal detection region having a positive charge cluster; when a negatively charged single molecule analyte in an electrolyte solution passes through said nanopore under the action of a driving voltage of the nanopore, a charge interaction between said positive charge cluster and the negatively charged single molecule analyte being capable of prolonging the residence time of said single molecule analyte within said nanopore.

8. The device according to claim 7, characterized in that, the charge number of said positive charge cluster can be controlled to regulate the residence time of said single molecule analyte within said nanopore.

9. The device according to claim 7, characterized in that, the driving voltage of said nanopore can be controlled to regulate the residence time of said single molecule analyte within said nanopore.

10. The device according to claim 7, characterized in that, the concentration of said electrolyte solution can be controlled to regulate the residence time of said single molecule analyte within said nanopore.

11. The device according to claim 7, characterized in that, said nanopore comprises a plurality of protein monomers.

12. The device according to claim 11, characterized in that, said protein monomer comprises any one of a-hemolysin, MspA, CsgG, OmpF.

13. The device according to claim 7, characterized in that, said single molecule analyte comprises nucleotides with different numbers of phosphates.

14. A nanopore detection method used for the nanopore detection device according to claim 7, characterized in that, the method comprises:
controlling the number of charges of positive charge cluster in the signal detection region of the nanopore to regulate the residence time of the negatively charged single molecule analyte within said nanopore as it passes through said nanopore.

15. The method according to claim 14, characterized in that, said controlling the number of charges of the positive charge cluster in the signal detection region of the nanopore to regulate the residence time of the negatively charged single molecule analyte within said nanopore as it passes through said nanopore comprises:
increasing the number of charges of the positive charge cluster in the signal detection region of said nanopore to prolong the residence time of the negatively charged single molecule analyte within said nanopore as it passes through said nanopore.

16. The method according to claim 14, characterized in that, the method further comprises:
controlling the driving voltage of said nanopore to regulate the residence time of the negatively charged single molecule analyte within said nanopore as it passes through said nanopore.

17. The method according to claim 16, characterized in that, said controlling the driving voltage of said nanopore to regulate the residence time of the negatively charged single molecule analyte within said nanopore as it passes through said nanopore comprises:
decreasing the driving voltage of said nanopore to prolong the residence time of the negatively charged single molecule analyte within said nanopore as it passes through said nanopore.

18. The method according to claim 14, characterized in that, the method further comprises:
controlling the concentration of said electrolyte solution to regulate the residence time of the negatively charged single molecule analyte within said nanopore as it passes through said nanopore.

19. The method according to claim 18, characterized in that, said controlling the concentration of said electrolyte solution to regulate the residence time of the negatively charged single molecule analyte within said nanopore as it passes through said nanopore comprises:
decreasing the concentration of said electrolyte solution to prolong the residence time of the negatively charged single molecule analyte within said nanopore as it passes through said nanopore.
